(19) United States
(12) Patent Application Publication
(10) Pub. No.: US 2016/0242819 A1
(43) Pub. Date: Aug. 25, 2016

(54) SPINAL IMPLANT SYSTEM AND METHOD

(71) Applicant: Warsaw Orthopedic, Inc., Warsaw, IN (US)

(72) Inventors: Joshua W. Simpson, Collierville, TN (US); Rex W. Armstrong, Cordova, TN (US); Dennis G. Crandall, Mesa, AZ (US); Maria-Rosa Padilla, Nesbit, MS (US); Gary S. Lindemann, Collierville, TN (US)

(21) Appl. No.: 14/631,363

(22) Filed: Feb. 25, 2015

(51) Int. Cl. A61B 17/70

(52) U.S. Cl. CPC A61B 17/7053; A61B 17/7049; A61B 2017/564

(57) ABSTRACT

A spinal construct comprises a member extending between a first end and a second end defining an opening configured for movement of the first end therethrough and disposal of the longitudinal member about spinal tissue. A malleable element is disposed with the first end. Systems and methods are disclosed.

### TECHNICAL HELD

The present disclosure generally relates to medical devices for the treatment of musculoskeletal disorders, and more particularly to a surgical system and method for correction of a spine disorder.

### BACKGROUND

Spinal pathologies and disorders such as scoliosis and other curvature abnormalities, kyphosis, degenerative disc disease, disc herniation, osteoporosis, spondylolisthesis, stenosis, tumor, and fracture may result from factors including trauma, disease and degenerative conditions caused by injury and aging. Spinal disorders typically result in symptoms including deformity, pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes correction, fusion, fixation, discectomy, laminectomy and implantable prosthetics. Correction treatments used for positioning and alignment may employ implants, such as vertebral rods, bone screws and sub-laminar wire, for stabilization of a treated section of a spine. This disclosure describes an improvement over these prior art technologies.

### SUMMARY

In one embodiment, a spinal construct comprises a member extending between a first end and a second end defining an opening configured for movement of the first end therethrough and disposal of the longitudinal member about spinal tissue. A malleable element is disposed with the first end. In some embodiments, systems and methods are disclosed.

### BRIEF DESCRIPTION OF THE DRAWINGS

The present disclosure will become more readily apparent from the specific description accompanied by the following drawings, in which:

FIG. 1 is a perspective view of one embodiment of components of a surgical system in accordance with the principles of the present disclosure;

FIG. 2 is a side view of one embodiment of components of a surgical system in accordance with the principles of the present disclosure;

FIG. 3A is a cross section view of the components shown in FIG. 2;

FIG. 3B is a cross section view of the components shown in FIG. 2;

FIG. 4 is a perspective view of one embodiment of components of a surgical system in accordance with the principles of the present disclosure;

FIG. 5 is a perspective view of one embodiment of components of a surgical system in accordance with the principles of the present disclosure;

FIG. 6 is a perspective view of the components shown in FIG. 2;

FIG. 7 is a plan view of one embodiment of components of a surgical system in accordance with the principles of the present disclosure;

FIG. 8 is a plan view of the components shown in FIG. 7 with parts separated;

FIG. 9 is a plan view of one embodiment of components of a surgical system in accordance with the principles of the present disclosure;

FIG. 10 is a plan view of one embodiment of components of a surgical system in accordance with the principles of the present disclosure;

FIG. 11 is a break away plan view of one embodiment of components of a surgical system in accordance with the principles of the present disclosure:

FIG. 12 is a break away plan view of one embodiment of components of a surgical system in accordance with the principles of the present disclosure;

FIG. 13 is a break away plan view of one embodiment of components of a surgical system in accordance with the principles of the present disclosure;

FIG. 14 is a break away plan view of one embodiment of components of a surgical system in accordance with the principles of the present disclosure;

FIG. 15 is a perspective view of one embodiment of components of a surgical system in accordance with the principles of the present disclosure;

FIG. 16 is a perspective view of one embodiment of components of a surgical system in accordance with the principles of the present disclosure disposed with vertebrae;

FIG. 17 is a perspective view of one embodiment of components of a surgical system in accordance with the principles of the present disclosure;

FIG. 18 is a break away plan view of one embodiment of components of a surgical system in accordance with the principles of the present disclosure;

FIG. 19 is a break away plan view of one embodiment of components of a surgical system in accordance with the principles of the present disclosure;

FIG. 20 is a break away perspective view of one embodiment of components of a surgical system in accordance with the principles of the present disclosure disposed with vertebrae;

FIG. 21 is a break away perspective view of the components and vertebrae shown in FIG. 20;

FIG. 22 is a break away perspective view of the components and vertebrae shown in FIG. 20;

FIG. 23 is a break away perspective view of the components and vertebrae shown in FIG. 20;

FIG. 24 is a break away perspective view of the components and vertebrae shown in FIG. 20;

FIG. 25 is a perspective view of one embodiment of components of a surgical system in accordance with the principles of the present disclosure;

FIG. 26 is a perspective view of the components shown in FIG. 25 with parts separated;

FIG. 27 is a cross section view of the components shown in FIG, 25;

FIG. 28 is a side view of one embodiment of components of a surgical system in accordance with the principles of the present disclosure;

FIG. 29 is a side view of one embodiment of components of a surgical system in accordance with the principles of the present disclosure;

FIG. 30 is a cross section view of the components shown in FIG. 29;

FIG. 31 is a side view of one embodiment of components of a surgical system in accordance with the principles of the present disclosure;

FIG. 32 is a side view of one embodiment of components of a surgical system in accordance with the principles of the present disclosure;

FIG. 33 is a side view of one embodiment of components of a surgical system in accordance with the principles of the present disclosure;

FIG. 34 is a perspective view of one embodiment of components of a surgical system in accordance with the principles of the present disclosure; and

FIG. 35 is a perspective view of the components shown in FIG. 34.

### DETAILED DESCRIPTION

The exemplary embodiments of a surgical system and related methods of use are discussed in terms of medical devices for the treatment of musculoskeletal disorders and more particularly, in terms of a surgical system and method for correction of a spine disorder. In some embodiments, the surgical system may be employed in applications for correction of deformities, such as scoliosis and kyphosis.

In some embodiments, the surgical system includes a spinal implant including a connector and a tether attached to a front portion of the connector. In one embodiment, the surgical system includes a set screw configured to apply a force to the spinal rod and the tether. In one embodiment, the set screw is configured to apply a force to the spinal rod causing the spinal rod to translate over the tether and fix the tether to the connector at two separate locking surfaces and/or points of contact. In one embodiment, the surgical system includes a tether having a loop that is configured for attachment to the connector and a loop configured for attachment with laminae.

In one embodiment, the surgical system includes a connector having two or more planar surfaces, such as, for example, flats and/or even surfaces configured to engage and/or retain the spinal rod. In one embodiment, the flats and/or even surfaces provide two or more separate locking surfaces and/or points of contact to facilitate gripping of a spinal rod. In one embodiment, the flats and/or even surfaces define one or more reliefs configured to prevent a spinal rod from undesirably engaging and/or pinching a tether at a sharp edge such that the tether is damaged and/or cut. In one embodiment, the connector includes four locking surfaces and/or points of contact with a spinal rod to facilitate fixation of the spinal rod and the tether with the connector.

In one embodiment, the surgical system includes a two piece connector. In one embodiment, the two piece connector facilitates clearance of a spinal rod. In one embodiment, the two piece connector includes a round edge and/or pin for attachment with a tether.

In some embodiments, the surgical system includes a connector configured for disposal of a spinal rod and a tether such that the spinal rod compresses the tether between the spinal rod and the connector to form a rigid connection by tightening of a set screw with the connector. In some embodiments, the surgical system includes a connector that allows for simultaneous contact of a spinal rod to the connector and is configured as a damp for a tether. In some embodiments, an angle of tether openings and contact areas can be varied during manufacture.

In one embodiment, the surgical system includes a multi-piece connector that includes a cover plate and fasteners. In one embodiment, the cover plate is configured to cover slots in the connector to facilitate capture of a tether. In one embodiment, the cover plate is attached to the connector during manufacture. In one embodiment, the surgical system includes a connector body having a threaded throughhole for a set screw, an upper tether slot, a middle portion to receive a spinal rod and a lower tether slot. In some embodiments, the surgical system is employed with a method that includes the steps of threading a tether from a top of the connector, into a middle portion of the connector and through a lower slot of the connector and passed out a bottom opening of the connector. In some embodiments, the method includes the steps of looping the tether around the spine, such as, for example, laminae and threading the tether in a reverse manner from the bottom of the connector. In one embodiment, the method includes the steps of looping a tether around the spine and ends of the tether are threaded into the connector through a lower tether slot, through a mid-portion of the connector and through an upper tether slot.

In one embodiment, the surgical system includes a set screw configured to capture a spinal rod by pressing the spinal rod against a tether such that tether presses against a portion of a wall of a connector. In one embodiment, a spinal rod is pressed against a second portion of a wall of a connector to rigidly capture the spinal rod and a tether to the connector. In one embodiment, the set screw provisionally fixes a tether allowing the tether to translate within slots in a connector for tensioning.

In one embodiment, the surgical system includes a connector having a first slot and a second slot disposed at a relative angular orientation. In some embodiments, the slots are disposed in an orientation to vary an amount of rod to connector body contact and tether to connector body contact. In some embodiments, the first and second slots are not in an in-line alignment or parallel relative to each other. In some embodiments, this configuration facilitates manufacture by cutting slots from a side portion of the connector and a cover plate is configured to cover an open side of the connector to capture the tether.

In one embodiment, the surgical system includes a sub-laminar tether configured for attachment with laminae and secures a vertebral body to a spinal rod, In some embodiments, an end of the tether, such as, for example, a paddle is configured for being passed safely and effectively about vertebrae. In some embodiments, the sub-laminar tether includes a malleable insertion paddle with a silicone coating. In some embodiments, the paddle may include an engagement element, such as, for example, a hole, knot or loop configured for attachment to a surgical instrument or nerve hook to engage the tether once passed under laminae.

In some embodiments, the tether includes a malleable insert configured to slide into the tether. In some embodiments, the malleable insert allows for flexibility and facilitates shaping and contouring. In some embodiments, the tether includes a metal insert attached to the tether via stitching, adhesive or heat sealing. In some embodiments, an end of the tether is coated with silicone to facilitate passage through patient anatomy. In some embodiments, the silicone coating is colored.

In some embodiments, the surgical system includes a tether that allows passing of the tether under the lamina without compromising the dura. In one embodiment, a magnetic tether paddle is provided to facilitate capture of the tether without using an invasive instrument. In one embodiment, a magnet is disposed with the tether paddle and connected with an instrument containing a ferro-magnetic metal. In one embodiment, the instrument includes a magnet and the tether paddle includes a ferro-magnetic metal. In one embodiment, the surgical system is employed with a method including the steps of passing the tether under laminae, positioning the instrument near a tip of the paddle, capturing the paddle with the instrument and pulling the paddle through an intervertebral space without interfering with the dura.

In some embodiments, the surgical system includes a connector that can be configured to selectively position an intersection of the tether slots relative to the contact surfaces in the throat of the connector to select the amount of surface contact area of the rod and the tether with the connector. In some embodiments, the intersection position is selectively oriented to affect system performance as the connector tightens the tether to prevent the tether from slipping and facilitates gripping the rod to resist and/or prevent the components of the system from slipping along the rod. In some embodiments, an increased surface contact area of the components results in an increased gripping performance between the components of the system.

In some embodiments, the surgical system includes a connector having a slot for disposal of the tether centrally positioned along a flat surface allowing surface contact with the tether and the rod. In some embodiments, the slot is disposed at a back edge of a flat surface allowing for rod contact and no tether contact.

In some embodiments, the surgical system includes a connector having a slot positioned along an upper portion of a flat surface providing for more tether surface contact and no rod contact. In some embodiments, the surgical system includes a connector having a slot disposed along an upper portion of a flat surface allowing for tether contact surface and a slot disposed along a bottom flat surface oriented to allow for more tether contact surface area and less rod contact.

In some embodiments, the surgical system is used with surgical navigation, such as, for example, fluoroscope or image guidance. In one embodiment, one or all of the components of the spinal implant system are disposable, peel-pack, pre-packed sterile devices. One or all of the components of the surgical system may be reusable. The surgical system may be configured as a kit with multiple sized and configured components.

In one embodiment, the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. In one embodiment, the present disclosure may be employed with other osteal and bone related applications, including those associated with diagnostics and therapeutics. In some embodiments, the disclosed surgical system and methods may be alternatively employed in a surgical treatment with a patient in a prone, supine position, lateral and/or employ various surgical approaches to the spine, including anterior, posterior, posterior mid-line, direct lateral, postero-lateral, and/or antero-lateral approaches, and in other body regions. The present disclosure may also be alternatively employed with procedures for treating the lumbar, cervical, thoracic, sacral and pelvic regions of a spinal column. The system and methods of the present disclosure may also be used on animals, bone models and other non-living substrates, such as, for example, in training, testing and demonstration.

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, in some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

As used in the specification and including the appended claims, “treating” or “treatment” of a disease or condition refers to performing a procedure that may include administering one or more drugs to a patient (human, normal or otherwise or other mammal), employing implantable devices, and/or employing instruments that treat the disease, such as, for example, micro discectomy instruments used to remove portions bulging or herniated discs and/or bone spurs, in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

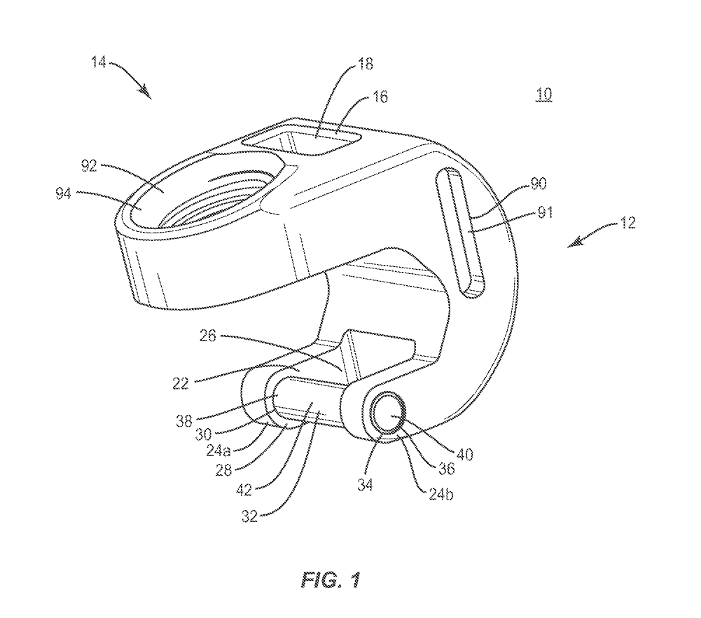

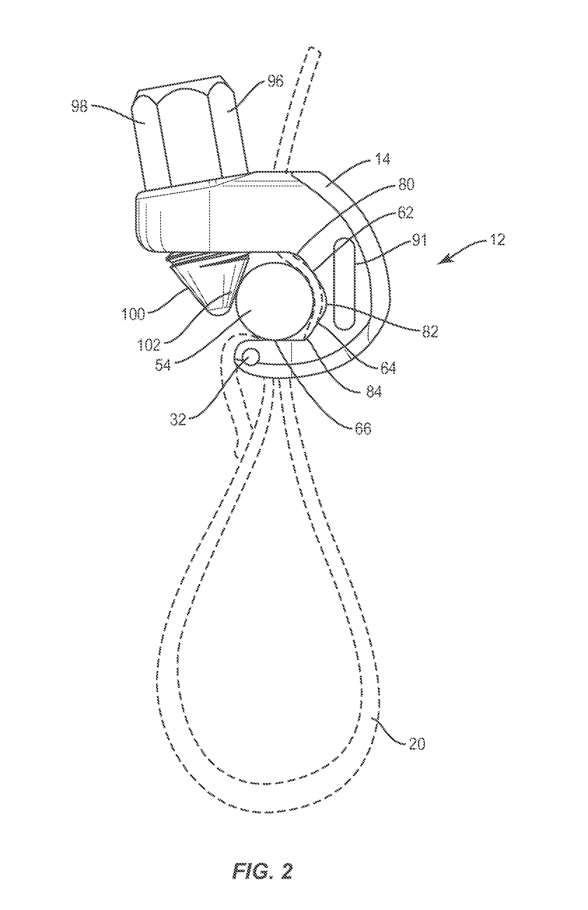

The following discussion includes a description of a surgical system and related methods of employing the surgical system in accordance with the principles of the present disclosure. Alternate embodiments are also disclosed. Reference is made in detail to exemplary embodiments of the present disclosure, which are illustrated in the accompanying figures. Turning to FIGS. 1-6, there are illustrated components of a surgical system, such as, for example, a spinal implant system 10.

The components of spinal implant system 10 can be fabricated from biologically acceptable materials suitable for medical applications, including metals, synthetic polymers, ceramics and bone material and/or their composites. For example, the components of spinal implant system 10, individually or collectively, can be fabricated from materials such as stainless steel alloys, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate such as hydroxyapatite (HA), corraline HA, biphasic calcium phosphate, tricalcium phosphate, or fluorapatite, tri-calcium phosphate (TOP), HA-TCP, calcium sulfate, or other resorbable polymers such as polyaetide, polyglycolide, polytyrosine carbonate, polycaroplaetohe and their combinations, biocompatible ceramics, mineralized collagen, bioactive glasses, porous metals, bone particles, bone fibers, morselized bone chips, bone morphogenetic proteins (BMP), such as BMP-2, BMP-4, BMP-7, rhBMP-2, or rhBMP-7, demineralized bone matrix (DBM), transforming growth factors (TGF, e.g., TGF-4), osteoblast cells, growth and differentiation factor (GDF), insulin-like growth factor 1, platelet-derived growth factor, fibroblast growth factor, or any combination thereof.

Various components of spinal implant system 10 may have material composites, including the above materials, to achieve various desired characteristics such as strength, rigidity, elasticity, compliance, biomechanical performance, durability and radiolucency or imaging preference. The components of spinal implant system 10, individually or collectively, may also be fabricated from a heterogeneous material such as a combination of two or more of the above-described materials. The components of spinal implant system 10 may be monolithically formed, integrally connected or include fastening elements and/or instruments, as described herein.

Spinal implant system 10 comprises a spinal implant, such as, for, example, a connector 12. Connector 12 includes a body 14 having a surface 16 that defines a cavity, such as, for example, an opening 18 configured for disposal of a member, such as, for example, a tether 20, as described herein. In some embodiments, opening 18 may have various cross section configurations, such as, for example, oval, oblong, triangular, rectangular, square, polygonal, irregular, uniform, non-uniform, variable and/or tapered. In some embodiments, surface 16 may include gripping elements or surfaces, such as, for example, rough, arcuate, undulating, mesh, porous, semi-porous, dimpled and/or textured to facilitate engagement with tether 20.

Body 14 includes a surface 22 that includes extensions 24a, 24b, Extensions 24a, 24b define a cavity, such as, for example, an opening 26 configured for disposal of tether 20. In some embodiments, opening 26 is disposed in alignment with opening 18. In some embodiments, opening 26 is disposed offset or staggered from opening 18. In some embodiments, opening 18 may have various cross section configurations, such as, for example, oval, oblong, triangular, rectangular, square, polygonal, irregular, uniform, non-uniform, variable and/or tapered. In some embodiments, surface 22 may include gripping elements or surfaces, such as, for example, rough, arcuate, undulating, mesh, porous, semi-porous, dimpled and/or textured to facilitate engagement with tether 20.

Extension 24a includes a surface 28 that defines a cavity 30. Cavity 30 is configured to receive a pin 32, as described herein. Extension 24b includes a surface 34 that defines a cavity 36. Cavity 36 is aligned with cavity 30. Cavity 36 is configured to receive pin 32, as described herein.

Pin 32 extends between an end 38 and an end 40. End 38 is disposed with cavity 30 and end 40 is disposed with cavity 36. Pin 32 has a cylindrical configuration and includes a surface 42 configured for attachment with tether 20, as described herein. In some embodiments, pin 32 may have alternate cross section configurations, such as, for example, oval, triangular, square, polygonal, irregular, uniform, non-uniform, offset, staggered, undulating, arcuate, variable and/or tapered.

Body 14 includes a surface 50 that defines a passageway 52. Passageway 52 has an oblong configuration and extends through body 14. In some embodiments, passageway 52 may have alternate cross section configurations, such as, for example, oval, cylindrical, triangular, square, polygonal, irregular, uniform, non-uniform, offset, staggered, undulating, arcuate, variable and/or tapered. Passageway 52 is configured for disposal of a longitudinal element, such as, for example, a spinal rod 54, as described herein, such that connector 12 can be mounted with spinal rod 54, as described herein.

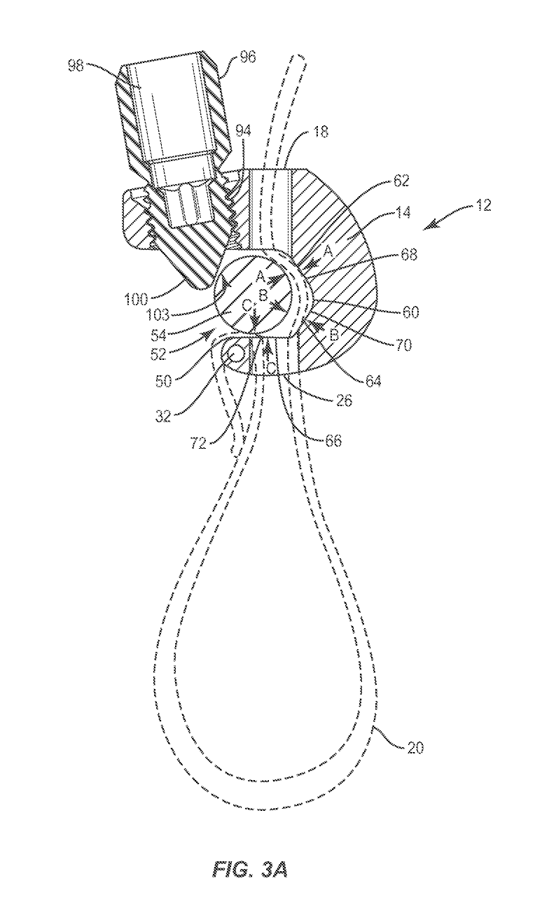

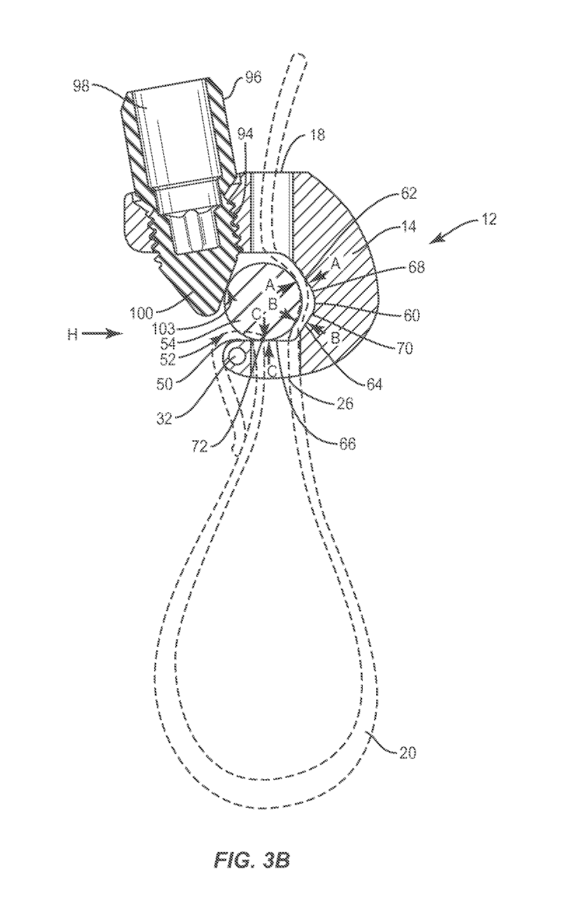

Surface 50 includes a wall 60 comprising a plurality of adjacent planar surfaces. Wall 60 includes a planar surface, such as, for example, a flat 62. Flat 62 defines an engagement surface and/or a lock surface 68 such that, with tether 20 disposed between spinal rod 54 and flat 62, spinal rod 54 is translated into engagement with tether 20. Spinal rod 54 engages tether 20 with flat 62 to fix tether 20 with connector 12 via compressive forces, applied at least in the direction shown by arrows A in FIGS. 3A and 3B. In some embodiments, the compressive forces applied adjacent surface 68 may be directed in various orientations, such as, for example, perpendicular, transverse and/or at angular orientations, such as acute or obtuse.

Wall 60 includes a planar surface, such as, for example, a flat 64 disposed adjacent flat 62. Fiat 64 defines an engagement surface and/or a lock surface 70 such that, with tether 20 disposed between spinal rod 54 and flat 64, spinal rod 54 is translated into engagement with tether 20. Spinal rod 54 engages tether 20 with flat 64 to fix tether 20 with connector 12 via compressive forces, applied at least in the direction shown by arrows B in FIGS. 3A and 38. In some embodiments, the compressive forces applied adjacent surface 70 may be directed in various orientations, such as, for example, perpendicular, transverse and/or at angular orientations, such as acute or obtuse.

Wall 60 includes a planar surface, such as, for example, a flat 66 disposed adjacent flat 64. Flat 66 defines an engagement surface and/or a lock surface 72 such that, with tether 20 disposed between spinal rod 54 and flat 66, spinal rod 54 is translated into engagement with tether 20. Spinal rod 54 engages tether 20 with flat 66 to fix tether 20 with connector 12 via compressive forces, applied at least in the direction shown by arrows C in FIGS. 3A and 3B. In some embodiments, the compressive forces applied adjacent surface 72 may be directed in various orientations, such as, for example, perpendicular, transverse and/or at angular orientations, such as acute or obtuse. In some embodiments, surface 50 may include gripping elements or surfaces, such as, for example, rough, arcuate, undulating, mesh, porous, semi-porous, dimpled and/or textured to facilitate engagement. In some embodiments, wall 60 includes a single planar surface. In some embodiments, wall 60 includes a combination of planar and arcuate surfaces.

In some embodiments, wall 60 defines a plurality of cavities between and adjacent flats 62, 64, 66 that prevent spinal rod 54 from undesirably engaging and/or pinching tether 20. Wall 60 includes a cavity, such as, for example, an arcuate relief 80 disposed adjacent flat 62. Relief 80 is recessed from surface 50 such that damage to tether 20 is resisted and/or prevented during engagement of tether 20 between spinal rod 54 and wall 60. Wall 60 includes a cavity, such as, for example, an arcuate relief 82 disposed adjacent and between flats 62, 64. Relief 82 is recessed from surface 50 such that damage to tether 20 is resisted and/or prevented during engagement of tether 20 between spinal rod 54 and wall 60. Wall 60 includes a cavity, such as, for example, an arcuate relief 84 disposed adjacent and between flats 64, 66. Relief 84 is recessed from surface 50 such that damage to tether 20 is resisted and/or prevented during engagement of tether 20 between spinal rod 54 and wall 60. In some embodiments, wall 60 can include one or more reliefs.

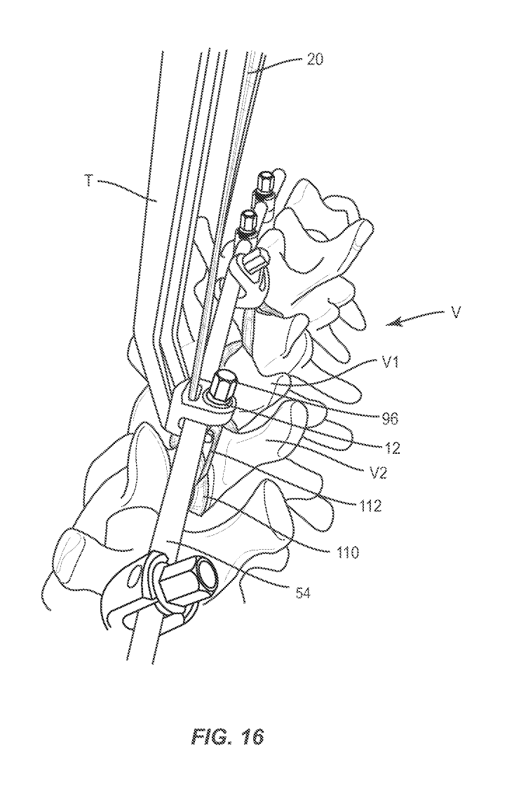

Body 14 includes a mating surface 90 that defines cavities, such as, for example, mating capture elements 91 configured to mate with a surgical instrument (for example, as shown in FIG. 16) to facilitate implant and manipulation of connector 12 and/or components of spinal implant system 10. In some embodiments, spinal implant system 10 may include one or a plurality of implant connectors spaced apart and disposed along spinal rod 54, which may be relatively disposed in a side by side, irregular, uniform, non-uniform, offset and/or staggered orientation or arrangement, along one or a plurality of spinal rods. In some embodiments, spinal rod 54 extends along one or a plurality of vertebra, as described herein, In some embodiments, spinal implant system 10 may include one or a plurality of spinal rods 54, which may be relatively disposed in a side by side, irregular, uniform, non-uniform, offset and/or staggered orientation or arrangement.

Body 14 includes a surface 92 that defines a cavity, such as, for example, an opening 94. Surface 92 is threaded and configured for disposal of a locking dement, such as, for example, a set screw 96. Set screw 96 extends between an end 98 and an end 100. End 98 is configured for engagement with a surgical instrument. In one embodiment, end 98 includes a hexagonal geometry configured for engagement with a similarly shaped tool, such as, for example, a driver. In some embodiments, end 98 has a cruciform, phillips, square, hexalobe, polygonal or star cross sectional configuration for disposal of a correspondingly shaped portion of a driver. End 100 includes a surface 102 configured for engagement with spinal rod 54. Surface 102 is tapered for engaging spinal rod 54. In some embodiments, as set screw 96 translates through opening 94, surface 102 translates spinal rod 54 into engagement with tether 20 to fix spinal rod 54 and tether 20 with connector 12, as described herein.

Set screw 96 is disposable between a non-locking orientation, as shown in FIG. 3A, such that spinal rod 54 and/or tether 20 are translatable relative to body 14 and a locked orientation, as shown in FIG. 3B, such that surface 102 translates spinal rod 54 into engagement with tether 20 to fix tether 20 with body 14. Engagement of surface 102 with spinal rod 54 at an engagement surface and/or a lock surface 103 causes translation of spinal rod 54 into engagement with tether 20, Tether 20 is compressed between spinal rod 54 and wall 60 including flats 62, 64, 66, as described herein, such that spinal rod 54 and tether 20 are locked and/or disposed in a fixed orientation with body 14 and relative to connector 12. In some embodiments, reliefs 80, 82, 84 prevent spinal rod 54 from pinching and/or cutting tether 20.

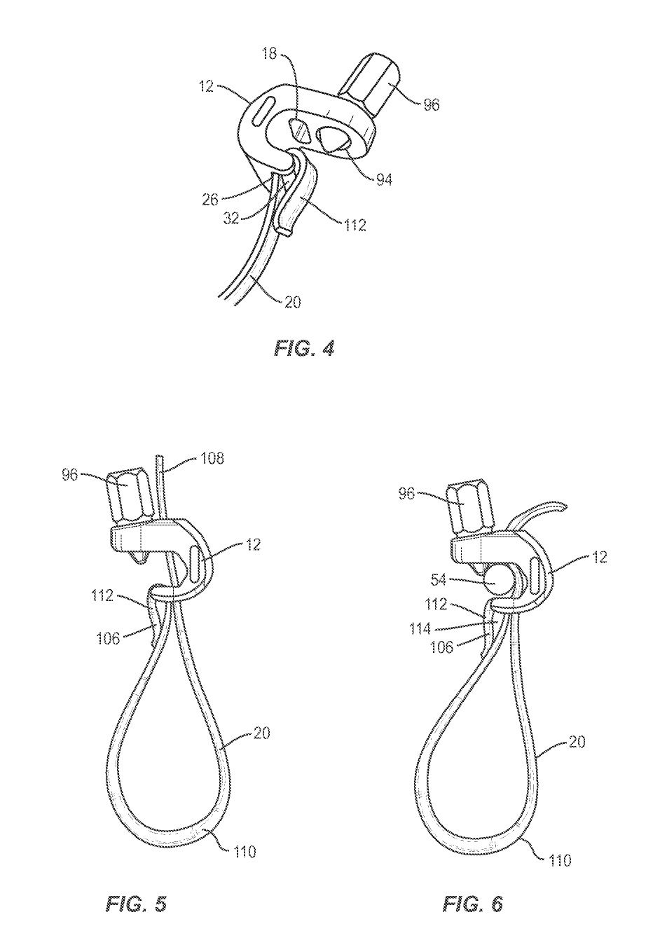

Tether 20 is a flexible longitudinal element that extends between an end 106 and an end 108, as shown in FIG. 5. Tether 20 is configured for engagement with connector 12, as described herein. In some embodiments, end 106 and end 108 form a loop 110 configured to surround all or a portion of tissue, such as, for example, laminae and/or spinal rod 54, as described herein. Tether 20 is configured for tensioning about a targeted portion of an anatomy of a body for attachment of tether 20 with the targeted portion of the anatomy, as described herein. In some embodiments, the targeted portion of the anatomy may include laminae, transverse process and/or pedicle regions of a vertebral level. In some embodiments, spinal implant system 10 may include one or a plurality of tethers 20, each tether being configured for disposal about a single and separate vertebral level. In some embodiments, a single vertebral level may include one or a plurality of tethers 20.

Tether 20 has a flexible configuration and may be fabricated from materials, such as, for example, fabric, silicone, polyurethane, silicone-polyurethane copolymers, polymeric rubbers, polyolefin rubbers, elastomers, rubbers, thermoplastic elastomers, thermoset elastomers and elastomeric composites. In one embodiment, the flexibility of tether 20 includes movement in a lateral or side to side direction and prevents expanding and/or extension in an axial direction upon tensioning and attachment with a targeted portion of the anatomy. In some embodiments, all or only a portion of tether 20 may have a semi-rigid, rigid or elastic configuration, and/or have elastic properties, similar to the material examples described above, such that tether 20 provides a selective amount of expansion and/or extension in an axial direction. In some embodiments, tether 20 may be compressible in an axial direction. Tether 20 can include a plurality of separately attachable or connectable portions or sections, such as bands or loops, or may be monolithically formed as a single continuous element.

Tether 20 can have a uniform thickness/diameter. In some embodiments, tether 20 may have various surface configurations, such as, for example, smooth and/or surface configurations to enhance fixation, such as, for example, rough, arcuate, undulating, porous, semi-porous, dimpled, polished and/or textured. In some embodiments, the thickness defined by tether 20 may be uniformly increasing or decreasing, or have alternate diameter dimensions along its length. In some embodiments, tether 20 may have various cross section configurations, such as, for example, oval, oblong, triangular, rectangular, square, polygonal, irregular, uniform, non-uniform, variable and/or tapered. In some embodiments, the surface of tether 20 may include engaging structures, such as, for example, barbs, raised elements and/or spikes to facilitate engagement with tissue of the targeted anatomy.

In some embodiments, tether 20 may have various lengths. In some embodiments, tether 20 may be braided, such as a rope, or include a plurality elongated elements to provide a predetermined force resistance. In some embodiments, tether 20 may be made from autograft and/or allograft, and be configured for resorbable or degradable applications. In one embodiment, tether 20 is a cadaver tendon. In one embodiment, tether 20 is a tendon that may be harvested, for example, from a patient or donor. In some embodiments, a tendon harvested from a patient may be affixed in remote locations with the patient's body.

In one embodiment, as shown in FIGS. 4-6, end 106 includes a loop 112 having an inner surface that defines an opening 114. An end most portion of end 106 is folded, wrapped and/or bent to connect with the surface of tether 20 to form loop 112. Opening 114 is configured for disposal of pin 32 such that tether 20 is attached with pin 32 and connector 12. In one embodiment, loop 114 is monolithically formed with end 106. In one embodiment, end 106 is stitched to a portion of tether 20 to form loop 112. In one embodiment, end 106 is connected to the surface of tether 20 with adhesive to form loop 112. In some embodiments, connector 12 is employed during a surgical treatment with tether 20 pre-attached. In some embodiments, end 106 and/or end 108 include non-looped, free ends.

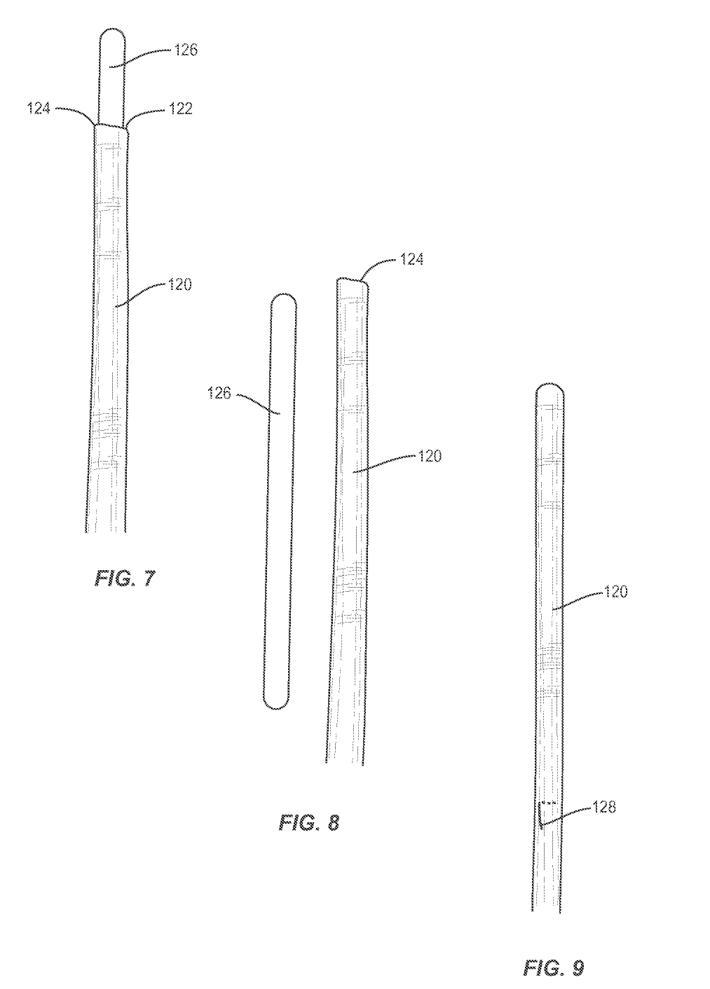

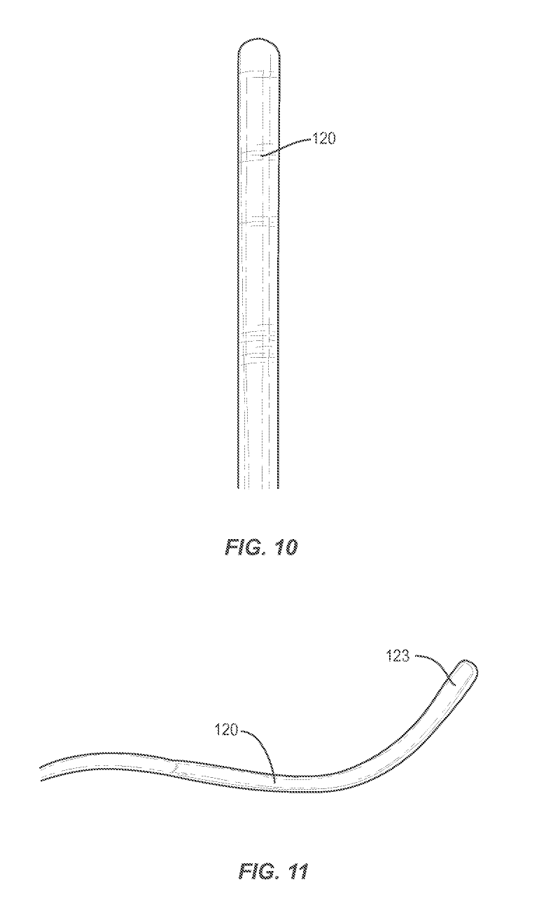

In some embodiments, as shown in FIGS. 7-10, a tether 120, similar to tether 20 described herein, includes an inner surface 122 that defines a cavity 124. Cavity 124 is configured for disposal of a metal insert 126, which is slid into tether 120 to form a malleable tether 120 such that tether 120 can be deformed into one or a plurality of alternate selected configurations. As such, tether 120 can be passed and/or guided through cavities defined by spinal tissue. As such, tether 120 resists and/or prevents non-desirable and/or harmful engagement with selected and/or sensitive anatomy of the spinal tissue. In some embodiments, insert 126 is attached to tether 120 via stitching 128, as shown in FIG. 9. In some embodiments, insert 126 is attached to tether 120 via adhesive and/or heat sealing. In one embodiment, as shown in FIG. 11, malleable tether 120 includes a malleable lead, such as, for example, a paddle 123 that can be deformed and/or contoured into one or a plurality of alternate selected configurations so that tether 120 can be passed and/or guided through cavities defined by spinal tissue.

In some embodiments, paddle 123 includes a malleable configuration such that paddle 123 can be passed and/or guided through cavities of spinal tissue to resist and/or prevent non-desirable and/or harmful engagement with selected and/or sensitive anatomy of the spinal tissue. In some embodiments, paddle 123 is configured for manipulation with a surgical instrument. In some embodiments, paddle 123 is soft and flexible and configured to pass through a sub-laminar cavity of vertebrae without adhering to dura matter of a spinal cord and/or surfaces of a lamina of a vertebral level. In some embodiments, all or only a portion of paddle 123 is fabricated from a low-friction material, such as, for example, silicone, polyurethane, silicone-polyurethane copolymers, polymeric rubbers, polyolefin rubbers, elastomers, rubbers, thermoplastic elastomers, thermoset elastomers and elastomeric composites. In some embodiments, all or only a portion of paddle 123 is fabricated from a semi-rigid, rigid or elastic configuration and/or have elastic properties, such as the elastic properties corresponding to the material examples described above. In some embodiments, all or only a portion of paddle 123 is fabricated from a material having a durometer in the range of approximately 30 A to 60 A under the ASTM D2240 type A scale. In one embodiment, paddle 123 comprises silicone having a durometer of 30 A to 60 A.

Paddle 123 includes an outer surface having an average surface roughness such that paddle 123 can be passed through the cavities of the spinal tissue and resist and/or prevent non-desirable and/or harmful adherence with selected and/or sensitive anatomy of the spinal tissue, for example, a lamina of a vertebral level and/or the dura matter. In some embodiments, all or only a portion of the surface of paddle 123 has an average surface roughness in a range of approximately 4 to 32 micro inches. In some embodiments, all or only a portion of the surface of paddle 123 has an average surface roughness in a range of approximately 4 to 16 micro inches. In some embodiments, all or only a portion of the surface of paddle 123 contacts spinal tissue, such as, for example, a lamina of a vertebral level and/or dura matter of a spinal cord, and the coefficient of kinetic friction of the surface of paddle 123 is in a range of approximately 0.04-0.50, such that the surface of paddle 123 slides along selected and/or sensitive anatomy of the spinal tissue with minimal resistance, adherence and/or sticking to the lamina and/or dura matter.

In some embodiments, paddle 123 has a length of approximately 40 mm to 60 mm so that paddle 123 can be passed under lamina of a particular vertebral level, In some embodiments, paddle 123 includes a tip having a blunt shape to resist and/or prevent non-desirable and/or harmful engagement with selected and/or sensitive anatomy of the spinal tissue. In some embodiments, paddle 123 has an arcuate configuration. In some embodiments, paddle 123 can have various shape configurations, such as, for example, oval, oblong, polygonal, irregular, uniform, non-uniform, variable and/or tapered.

In some embodiments, paddle 123 includes a substantially uniform thickness. In some embodiments, paddle 123 is variously shaped, such as, for example, oval, oblong, triangular, square, polygonal, irregular, uniform, non-uniform, offset, staggered, undulating, arcuate, variable and/or tapered. In some embodiments, paddle 123 may have various cross section configurations, such as, for example, oval, oblong, triangular, rectangular, square, polygonal, irregular, uniform, non-uniform, variable, tubular and/or tapered.

In some embodiments, paddle 123 includes a cavity configured for disposal of a longitudinal member, such as, for example, tether 120. In one embodiment, paddle 123 is permanently molded with tether 120. In some embodiments, paddle 123 is detachably or removably engaged with tether 120. In some embodiments, paddle 123 can be variously connected with tether 120, such as, for example, monolithic, integral connection, frictional engagement, threaded engagement, mutual grooves, screws, adhesive, nails, barbs and/or raised element.

In some embodiments, paddle 123 includes a uniform width. In one embodiment, paddle 123 has a width of approximately 6 mm. In some embodiments, paddle 123 includes a substantially even face and being oriented in a first direction and a substantially even face and being oriented in a second direction opposite the first direction. In some embodiments, paddle 123 has a substantially uniform thickness along its length. In some embodiments, the thickness of paddle 123 may be non-uniform, uniformly increasing or uniformly decreasing. In some embodiments, paddle 123 has a thickness of approximately 2 mm. In some embodiments, one of the faces of paddle 123 is concavely curved and the opposite faces is convexly curved such that paddle 123 is arcuate along its length. In some embodiments, paddle 123 has an arcuate configuration having a radius of curvature. In some embodiments, paddle 123 has a radius of curvature in a range of approximately 20 mm to 100 mm. In some embodiments, tether 120 is substantially flexible relative to paddle 123.

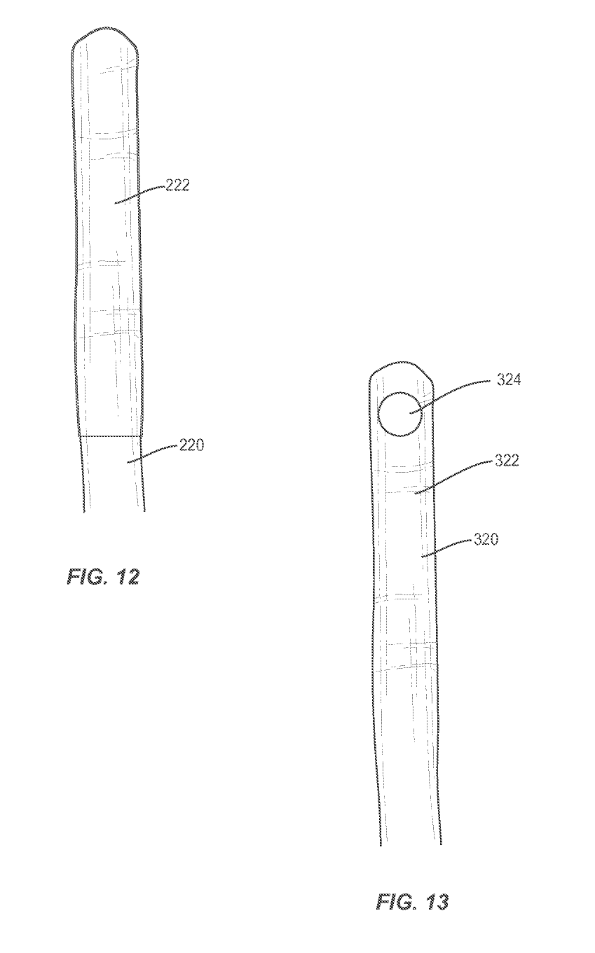

In one embodiment, as shown in FIG. 12, all or only a portion of a tether 220, similar to tether 20 described herein, includes a coating 222. In some embodiments, coating 222 may include polyurethane, silicone-polyurethane copolymers, polymeric rubbers, polyolefin rubbers, elastomers, rubbers, thermoplastic elastomers, thermoset elastomers and/or elastomeric composites. In some embodiments, coating 222 includes visual indicia, such as, for example, coloration for identification during selection, a treatment and/or to facilitate manipulation.

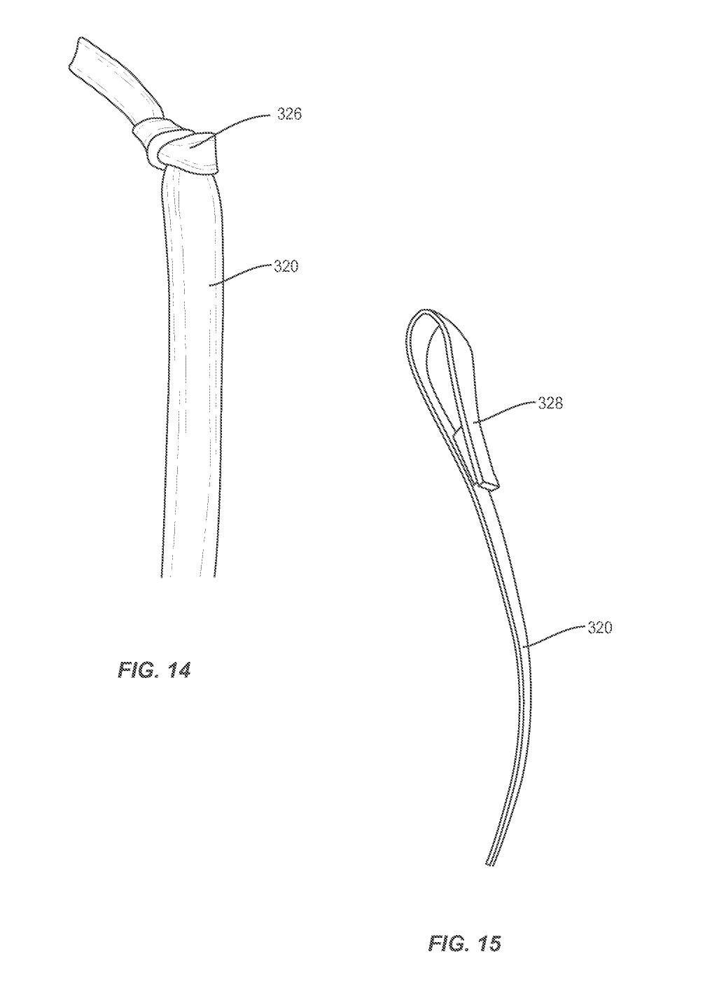

In one embodiment, as shown in FIG. 13, a tether 320, similar to tether 20 described herein, includes a paddle 322 having an opening 324 configured for connection to a portion of tether 320, a separate tether and/or spinal implant, and/or a surgical instrument. In some embodiments, opening 324 connects with a surgical instrument, such as, for example a nerve hook. In one embodiment, as shown in FIG. 14, tether 320 includes a knot 326 configured for connection to a portion of tether 320, a separate tether and/or spinal implant, and/or a surgical instrument. In one embodiment, as shown in FIG. 15, tether 320 includes a loop 328 configured for connection to a portion of tether 320, a separate tether and/or spinal implant, and/or a surgical instrument.

In assembly, operation and use, spinal implant system 10, similar to the systems and methods described herein, is employed with a surgical procedure, such as, for example, a correction treatment of an affected portion of a spine, for example, a correction treatment to treat adolescent idiopathic scoliosis and/or Scheuermann's kyphosis of a spine. In some embodiments, one or all of the components of spinal implant system 10 can be delivered or implanted as a pre-assembled device or can be assembled in situ. Spinal implant system 10 may be completely or partially revised, removed or replaced.

In use, to treat a selected section of vertebrae V, as shown in FIG. 16, a medical practitioner obtains access to a surgical site including vertebrae V in any appropriate manner, such as through incision and retraction of tissues. In some embodiments, spinal implant system 10 can be used in any existing surgical method or technique including open surgery, mini-open surgery, minimally invasive surgery and percutaneous surgical implantation, whereby vertebrae V is accessed through a mini-incision, or a sleeve that provides a protected passageway to the area. Once access to the surgical site is obtained, the particular surgical procedure can be performed for treating the spine disorder.

An incision is made in the body of a patient and a cutting instrument (not shown) creates a surgical pathway for implantation of components of spinal implant system 10. A preparation instrument (not shown) can be employed to prepare tissue surfaces of vertebrae V, as well as for aspiration and irrigation of a surgical region.

Tether 20 is attached with connector 12, as described herein. Loop 112 is connected with pin 32. Connection of tether 20 with pin 32 causes tether 20 to extend from opening 26. Tether 20 is delivered along the surgical pathway to a surgical site adjacent vertebrae V. Tether 20 is disposed with vertebrae V and threaded with the openings and passageways of connector 12, as described herein, to capture selected spinal tissue of vertebrae V.

In one embodiment, loop 110 is disposed about a transverse process of a vertebra V2 by passing end 108 continuously about the transverse process. End 108 is inserted into opening 26 to form loop 110. Loop 110 is fixed and/or attached with the transverse process and/or lamina. Tether 20 is threaded through opening 26, passageway 52 and opening 18. Spinal rod 54 is disposed with passageway 52 such that tether 20 is disposed between spinal rod 54 and wall 60, as described herein, and reduced with connector 12 to vertebrae V. Set screw 96 is engaged with opening 94. End 98 is engaged with a surgical instrument, such as, for example, a driver to advance set screw 96 into opening 94 in a non-locking orientation, as described herein, such that tether 20 is translatable relative to body 14 to tension tether 20 about vertebra V2.

A surgical instrument, such as, for example, a tensioner T is disposed adjacent connector 12. Tensioner T is aligned and engaged with mating capture elements 91 for releasable fixation and/or provisional fixation of tensioner T with connector 12. Tensioner T is actuated to tension tether 20 about vertebra V2 for connecting spinal rod 54 with vertebrae V. In some embodiments, the tension and/or tensile force applied to tether 20 and/or corrective forces applied to vertebrae V can be increased and/or decreased by tensioner T.

As set screw 96 is rotated for translation to a locked orientation, as described herein, such that surface 102 engages spinal rod 54. Surface 102 translates spinal rod 54 into engagement with tether 20, which is disposed between spinal rod 54 and wall 60. Tether 20 is compressed between spinal rod 54 and wall 60 including flats 62, 64, 66, as described herein, such that spinal rod 54 and tether 20 are locked and/or disposed in a fixed orientation with body 14 and relative to connector 12 adjacent vertebra V2. Reliefs 80, 82, 84 prevent spinal rod 54 from pinching and/or cutting tether 20. This configuration tensions tether 20 about vertebra V2 and tensions the spinal construct for attachment with vertebrae V and/or to apply corrective treatment to vertebrae V.

In some embodiments, spinal implant system 10 includes a second spinal rod (not shown) delivered along the surgical pathway to the surgical site adjacent a contra-lateral side of vertebrae V. The second spinal rod is connected with the contra-lateral side of vertebrae V via one or more tethers 20, similar to spinal rod 54 described herein. Spinal rod 54 and the second spinal rod are fixed with vertebrae V in a side by side orientation and/or a bi-lateral arrangement to stabilize vertebrae V and affect growth for a correction treatment to treat spine pathologies, as described herein.

Upon completion of the procedure, the surgical instruments, assemblies and non-implanted components of spinal implant system 10 are removed from the surgical site and the incision is closed. One or more of the components of spinal implant system 10 can be made of radiolucent materials such as polymers. Radiomarkers may be included for identification under x-ray, fluoroscopy, CT or other imaging techniques. In some embodiments, the use of surgical navigation, microsurgical and image guided technologies may be employed to access, view and repair spinal deterioration or damage, with the aid of spinal implant system 10.

In some embodiments, spinal implant system 10 includes an agent, which may be disposed, packed, coated or layered within, on or about the components and/or surfaces of spinal implant system 10. In some embodiments, the agent may include bone growth promoting material, such as, for example, bone graft to enhance fixation of the bone fasteners with vertebrae. In some embodiments, the agent may include one or a plurality of therapeutic agents and/or pharmacological agents for release, including sustained release, to treat, for example, pain, inflammation and degeneration.

In some embodiments, the components of spinal implant system 10 may be employed to treat progressive idiopathic scoliosis with or without sagittal deformity in either infantile or juvenile patients, including but not limited to prepubescent children, adolescents from 10-12 years old with continued growth potential, and/or older children whose growth spurt is late or who otherwise retain growth potential. In some embodiments, the components of spinal implant system 10 may be used to prevent or minimize curve progression in individuals of various ages.

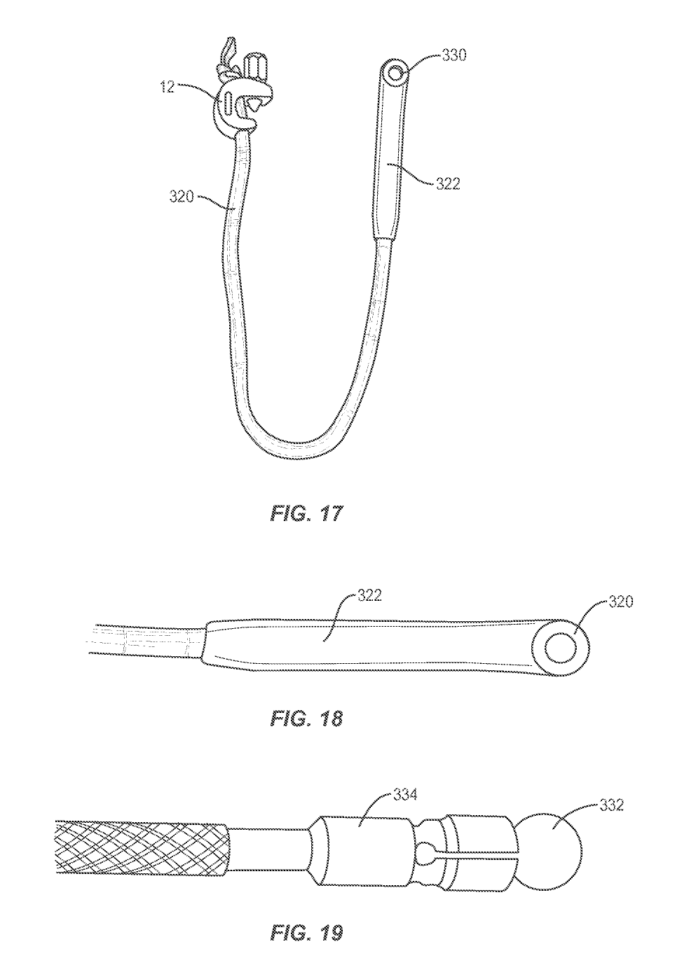

In some embodiments, as shown in FIGS. 17-19, paddle 322 includes a magnet 330. Paddle 322 is employed with a surgical instrument 334, as shown in FIG. 19, for guiding tether 320 during a surgical treatment, as described herein. In some embodiments, paddle 322 is disposed with cavities of spinal tissue, for example, adjacent laminae. Surgical instrument 334 includes a ferro-magnetic portion 332 that is disposed adjacent paddle 322 and magnetically attracts magnet 330 to draw paddle 322 via magnetic attraction along a selected pathway adjacent the spinal tissue. As such, paddle 322 is selectively drawn through tissue so that tether 320 can be passed and/or guided through cavities defined by spinal tissue. Surgical instrument 334 draws paddle 322 along the selected pathway such that non-desirable and/or harmful engagement of tether 320 with selected and/or sensitive anatomy of the spinal tissue is resisted and/or prevented.

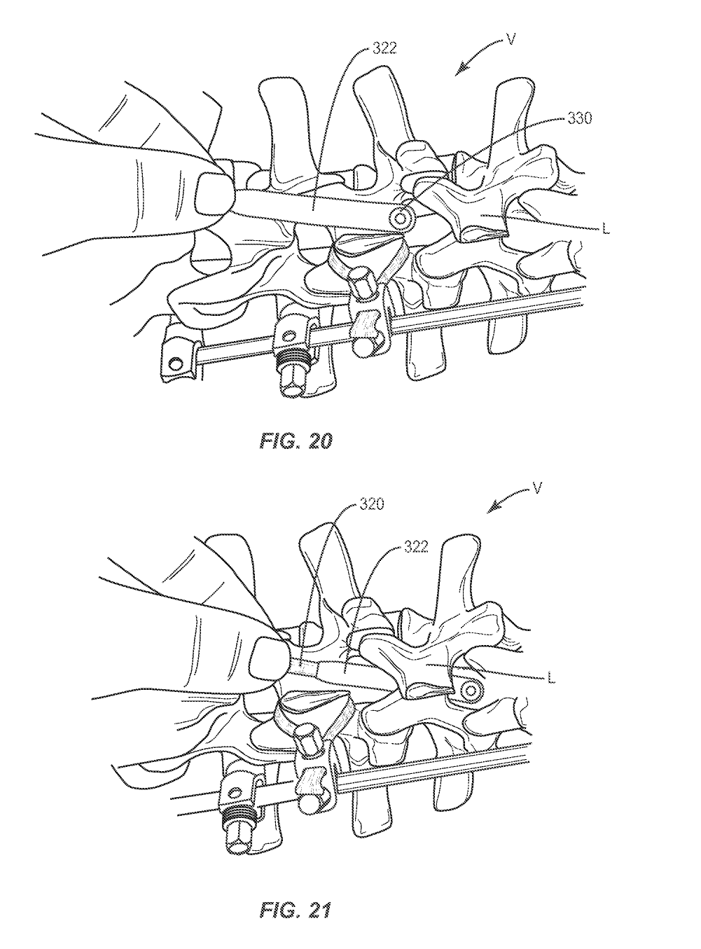

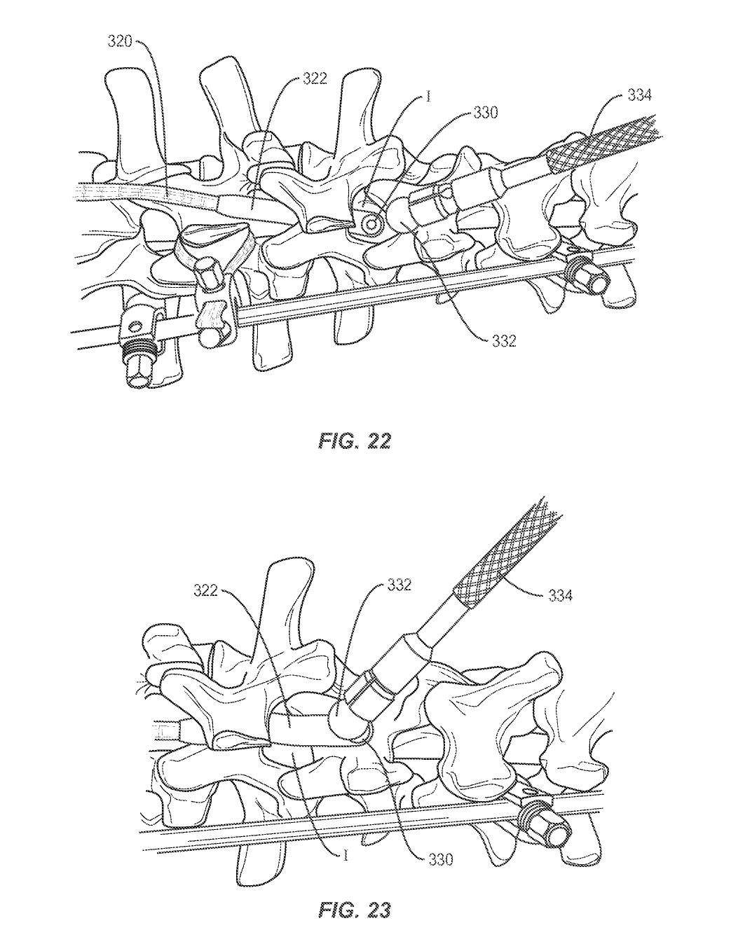

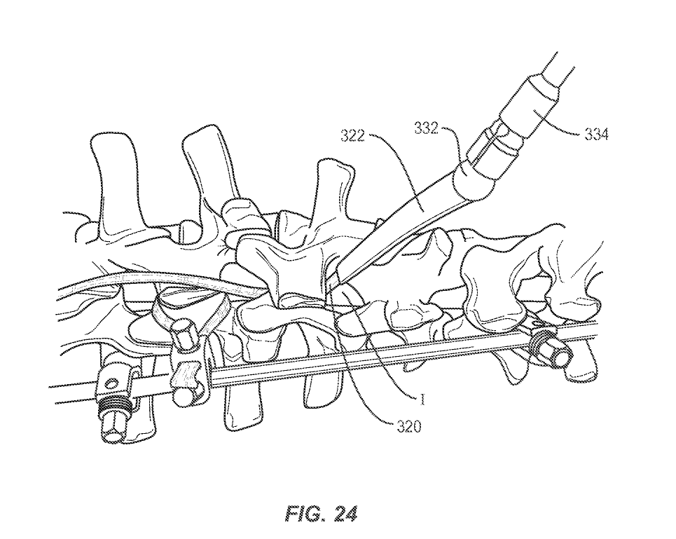

In use, as shown in FIGS. 20-24, spinal implant system 10 including tether 320 having magnet 330, similar to the systems and methods described herein, is employed with a surgical procedure, as described herein. For example, as shown in FIGS. 20 and 21, paddle 322 including magnet 330 is passed under a lamina L of vertebrae V. Portion 332 is positioned adjacent the tip of paddle 322 including magnet 330, as shown in FIG. 22. Portion 332 magnetically attracts magnet 330 into contact and instrument 334 captures paddle 322, as shown in FIG. 23. Instrument 334 draws paddle 322 and tether 320 through an intervertebral space I of vertebrae V, as shown in FIG. 24. In some embodiments, paddle 322 includes a ferro-magnetic metal portion and surgical instrument 334 includes a magnet.

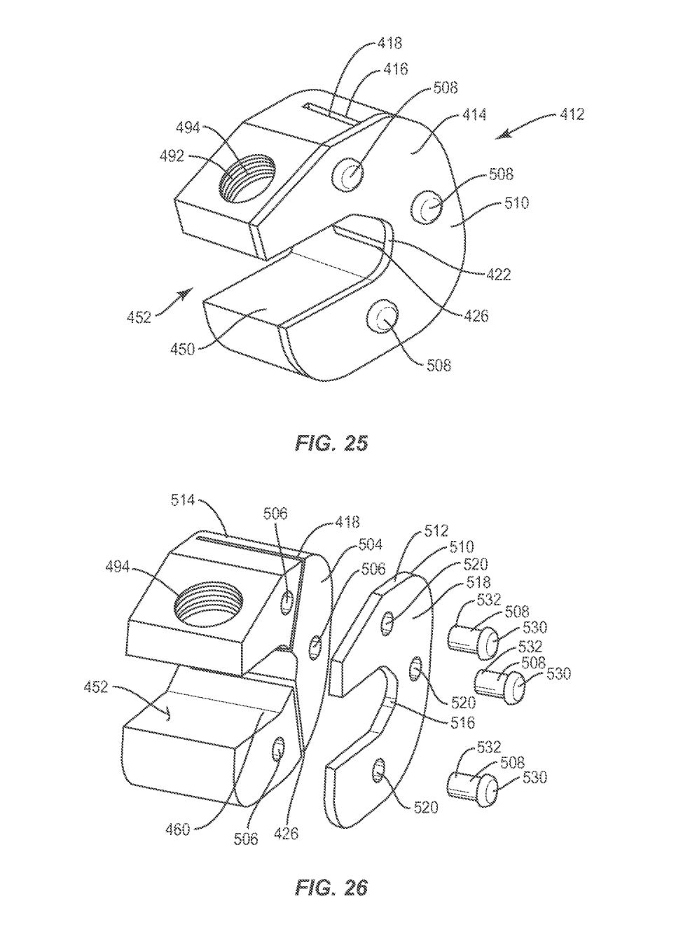

In one embodiment, as shown in FIGS. 25-28, spinal implant system 10, similar to the systems and methods described herein, comprises a connector 412, similar to connector 12 described herein. Connector 412 includes a body 414 having a surface 416 that defines a cavity, such as, for example, an opening 418 configured for disposal of tether 20, as described herein.

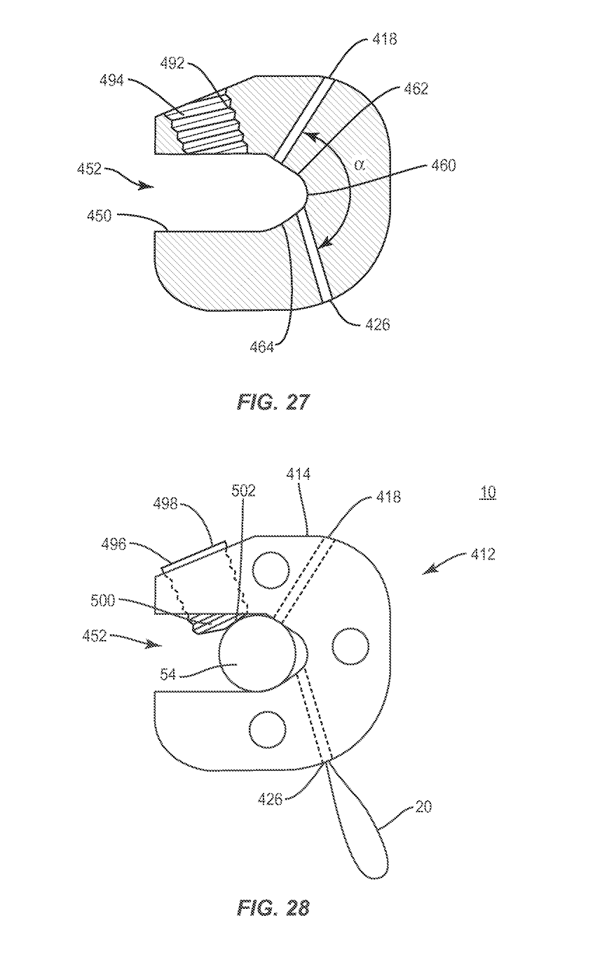

Body 414 includes a surface 422 that defines a cavity, such as, for example, an opening 426 configured for disposal of tether 20. Opening 418 is oriented at an angle a relative to opening 426, as shown in FIG. 27. In some embodiments, angle a can be altered to vary the amount of spinal rod 54 to connector 412 contact and/or tether 20 to connector 412 contact. In some embodiments, angle a may include an angle in a range of 0 through 270 degrees.

Body 414 includes a surface 450 that defines a passageway 452. Passageway 452 has an oblong configuration and extends through body 414. Passageway 452 is configured for disposal of spinal rod 54, as described herein, such that connector 412 can be mounted with spinal rod 54, as described herein.

Surface 450 defines a wall 460, similar to wall 60 described herein. Wall 460 includes planar surfaces, such as, for example, flats 462, 464, similar to flats 62, 64 described herein. Body 414 includes a surface 492 that defines an opening 494. Surface 492 is threaded and configured for disposal of a set screw 496. Set screw 496 extends between an end 498 and an end 500. End 498 is configured for engagement with a surgical driver. End 500 includes a lock surface 502 configured for engagement with spinal rod 54.

Set screw 496 is disposable between a non-locking orientation such that tether 20 is translatable relative to body 414 and a locked orientation such that lock surface 502 of set screw 496 translates spinal rod 54 into engagement with tether 20 to selectively fix tether 20 with body 414, similar to connector 12 described herein.

Body 414 includes a surface 504 that defines openings 506. Openings 506 are configured for engagement with locking elements, such as, for example, fasteners 508, as described herein. Openings 506 are disposed equidistant about surface 504. In some embodiments openings 506 are disposed offset and/or staggered about surface 504.

A capture element, such as, for example, a plate 510 includes a surface 512 aligned with a lateral surface 514 of body 414. Plate 510 includes a wall 516 configured to align with wall 460. Alignment of surfaces 512, 514 and walls 516, 460 forms connector 412. Plate 510 includes a surface 518 that defines openings 520. Openings 520 are configured to align with openings 506 for engagement with fasteners 508. Plate 510 is configured to cover openings 418, 426 to fix tether 20 to body 414. Each of fasteners 508 includes a head portion 530 and a shaft portion 532. Shaft portion 532 is configured for disposal with openings 506, 520. Head portions 530 engage plate 510 to secure plate 510 with body 414. In some embodiments, plate 510 captures tether 20 with connector 414 such that tether 20 can be fixed with connector 412, as described herein. In some embodiments, plate 510 is assembled with connector 412 during manufacture and prior to threading of tether 20 with body 414, as described herein.

In use, as shown in FIG. 28, spinal implant system 10 including tether 20 and connector 412, similar to the systems and methods described herein, is employed with a surgical procedure, as described herein. End 106 of tether 20 is threaded through opening 418 into passageway 452 and through opening 426 such that end 106 extends past surface 422 of opening 426. End 106 is looped around lamina, as described herein, and then threaded in a reverse manner through opening 426, through passageway 452 and through opening 418. In some embodiments, tether 20 can be threaded through connector 412 in a reverse configuration. In some embodiments, connector 412 includes a pin, similar to pin 32 described herein, and tether 20 includes a loop, similar to loop 112 described herein, such that the tether loop is attached to connector 412 and tether 20 is connected with the lamina and threaded through opening 426, passageway 452 and opening 418.

Tether 20 extends past surface 416 of opening 418 for attachment to an instrument for tensioning of tether 20, as described herein. Plate 510 captures tether 20 within openings 418, 426. In some embodiments, body 414 includes mating capture elements, similar to mating capture elements 91 described herein, configured to mate with a surgical instrument, as described herein.

Set screw 496 is rotated for translation to a locked orientation, as described herein, such that surface 502 engages spinal rod 54. Surface 502 translates spinal rod 54 into engagement with tether 20, which is disposed between spinal rod 54 and wall 460. Tether 20 is compressed between spinal rod 54 and wall 460 including flats 462, 464, similar to that described herein, such that spinal rod 54 and tether 20 are locked and/or disposed in a fixed orientation with body 414 and relative to connector 412.

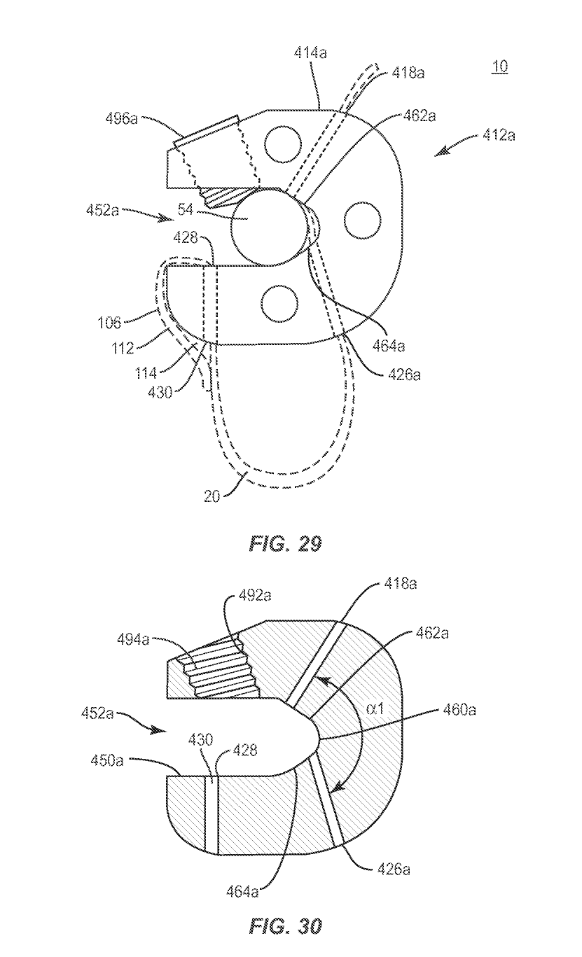

In one embodiment, as shown in FIGS. 29 and 30, spinal implant system 10, similar to the systems and methods described herein, comprises a connector 412a, similar to connector 412 described herein. Connector 412a includes a body 414a having an opening 418a configured for disposal of tether 20, as described herein. Body 414a includes an opening 426a configured for disposal of tether 20. Opening 418a is oriented at an angle α1 relative to opening 426a, as shown in FIG. 30. In some embodiments, angle α1 can be altered to vary the amount of spinal rod 54 to connector 412a contact and/or tether 20 to connector 412a contact. In some embodiments, angle α1 may include an angle in a range of 0 through 270 degrees.

Body 414a includes a surface 428 that defines an opening 430 configured for disposal of tether 20. End 106 of tether 20, as described herein, is folded, wrapped and/or bent to connect with surface 428. End 106 includes a loop 112 having an inner surface that defines an opening 114. Opening 114 is configured for disposal of surface 428 such that tether 20 is attached with connector 412a.

Body 414a includes a surface 450a that defines a passageway 452a. Passageway 452a has an oblong configuration and extends through body 414a. Passageway 452a is configured for disposal of spinal rod 54, as described herein, such that connector 412a can be mounted with spinal rod 54, as described herein.

Surface 450a defines a wall 460a, similar to wall 60 described herein. Wall 460a includes planar surfaces, such as, for example, flats 462a, 464a, similar to flats 62, 64 described herein. Body 414a includes a surface 492a that defines an opening 494a. Surface 492a is threaded and configured for disposal of a set screw 496a, similar to set screw 96, described herein.

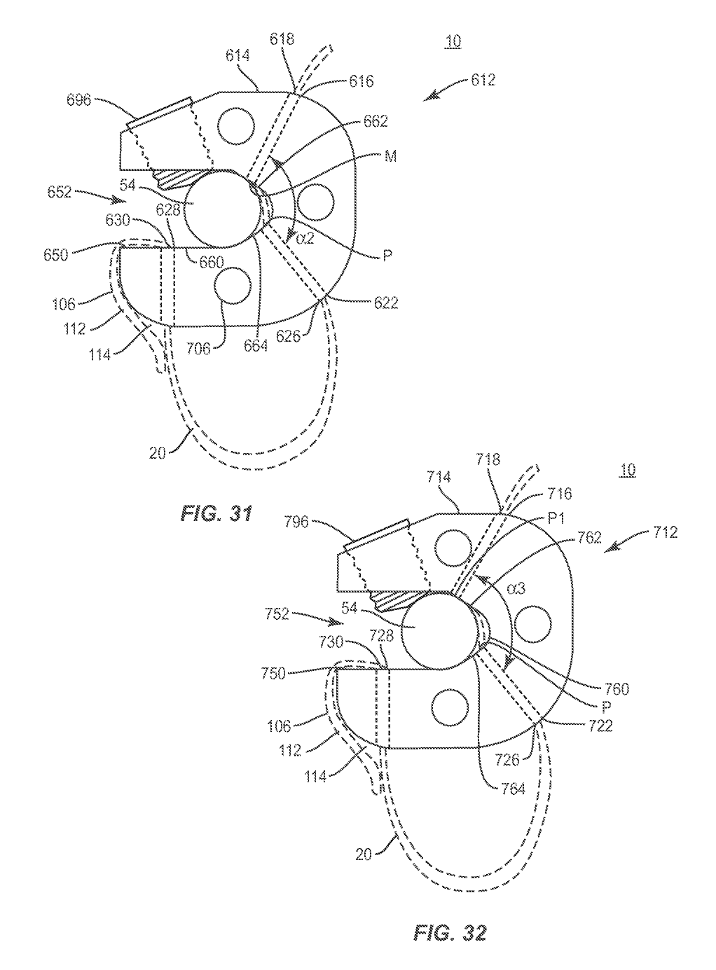

In one embodiment, as shown in FIG. 31, spinal implant system 10, similar to the systems and methods described herein, comprises a connector 612, similar to connector 412 described herein. Connector 612 includes a body 614 having a surface 616 that defines an opening 618 configured for disposal of tether 20, as described herein.

Body 614 includes a surface 622 that defines an opening 626 configured for disposal of tether 20. Opening 618 is oriented at an angle α2 relative to opening 626. Body 614 includes a surface 628 that defines an opening 630 configured for disposal of tether 20. End 106 of tether 20, as described herein, is folded, wrapped and/or bent to connect with surface 628. End 106 includes a loop 112 having an inner surface that defines an opening 114. Opening 114 is configured for disposal of surface 628 such that tether 20 is attached with connector 612.

Body 614 includes a surface 650 that defines a passageway 652. Passageway 652 has an oblong configuration and extends through body 614. Passageway 652 is configured for disposal of spinal rod 54, as described herein, such that connector 612 can be mounted with spinal rod 54, as described herein.

Surface 650 defines a wall 660, similar to wall 60 described herein. Wall 660 includes planar surfaces, such as, for example, flats 662, 664, similar to flats 62, 64 described herein. Opening 618 is disposed at a midpoint M of flat 662. Positioning opening 618 at midpoint M provides surface contact with tether 20 and contact with spinal rod 54. Opening 626 is positioned at a point P oriented towards a back edge of fiat 664. Orienting opening 626 towards the back edge of flat 664 provides an increased surface contact with tether 20 and decreases the surface contact with spinal rod 54. The intersection of openings 618, 626 with flats 663, 664 alters the surface contact area of spinal rod 54 and tether 20 to connector 612. Increased surface contact between spinal rod 54 and tether 20 area facilitates gripping with connector 612.

Set screw 696 is disposable between a non-locking orientation such that tether 20 is translatable relative to body 614 and a locked orientation to selectively fix tether 20 with body 614, similar to connector 12 described herein.

In one embodiment, as shown in FIG. 32, spinal implant system 10, similar to the systems and methods described herein, comprises a connector 712, similar to connector 412 described herein. Connector 712 includes a body 714 having a surface 716 that defines an opening 718 configured for disposal of tether 20, as described herein.

Body 714 includes a surface 722 that defines an opening 726 configured for disposal of tether 20. Opening 718 is oriented at an angle α3 relative to opening 726. Body 714 includes a surface 728 that defines an opening 730 configured for disposal of tether 20. End 106 of tether 20, as described herein, is folded, wrapped and/or bent to connect with surface 728 connector 612. End 106 includes a loop 112 having an inner surface that defines an opening 114. Opening 114 is configured for disposal of surface 728 such that tether 20 is attached with connector 712.

Body 714 includes a surface 750 that defines a passageway 752. Passageway 752 has an oblong configuration and extends through body 714. Passageway 752 is configured for disposal of spinal rod 54, as described herein, such that connector 712 can be mounted with spinal rod 54, as described herein.

Surface 750 defines a wall 760, similar to wall 60 described herein. Wall 760 includes planar surfaces, such as, for example, flats 762, 764, similar to flats 62, 64 described herein. Opening 718 is disposed at a point P1 of flat 762. Point P1 is oriented at an upper portion of flat 762 providing an increased surface contact with tether 20. Opening 726 is positioned at a point P oriented towards a back edge of flat 764 to prevent surface contact with spinal rod 54.

Set screw 796 is disposable between a non-locking orientation such that tether 20 is translatable relative to body 714 and a locked orientation to selectively fix tether 20 with body 614, similar to connector 12 described herein.

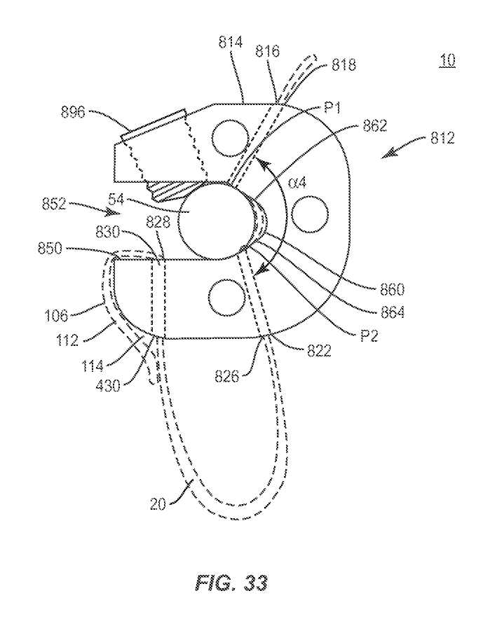

In one embodiment, as shown in FIG. 33, spinal implant system 10, similar to the systems and methods described herein, comprises a connector 812, similar to connector 412 described herein. Connector 812 includes a body 814 having a surface 816 that defines an opening 818 configured for disposal of tether 20, as described herein.

Body 814 includes a surface 822 that defines an opening 826 configured for disposal of tether 20. Opening 818 is oriented at an angle α4 relative to opening 826. Body 814 includes a surface 828 that defines an opening 830 configured for disposal of tether 20. End 106 of tether 20, as described herein, is folded, wrapped and/or bent to connect with surface 828. End 106 includes a loop 112 having an inner surface that defines an opening 114. Opening 114 is configured for disposal of surface 828 such that tether 20 is attached with connector 812.

Body 814 includes a surface 850 that defines a passageway 852. Passageway 852 has an oblong configuration and extends through body 814. Passageway 852 is configured for disposal of spinal rod 54, as described herein, such that connector 812 can be mounted with spinal rod 54, as described herein.

Surface 850 defines a wall 860, similar to wall 60 described herein. Wall 860 includes planar surfaces, such as, for example, flats 862, 864, similar to flats 62, 64 described herein. Opening 818 is disposed at a point P1 of flat 862. Point P1 is oriented at an upper portion of flat 862 providing an increased surface contact with tether 20. Opening 826 is positioned at a point P2 oriented towards a forward edge of flat 864 allowing for increased surface contact with tether 20.

Set screw 896 is disposable between a non-locking orientation such that tether 20 is translatable relative to body 814 and a locked orientation to selectively fix tether 20 with body 814, similar to connector 12 described herein.

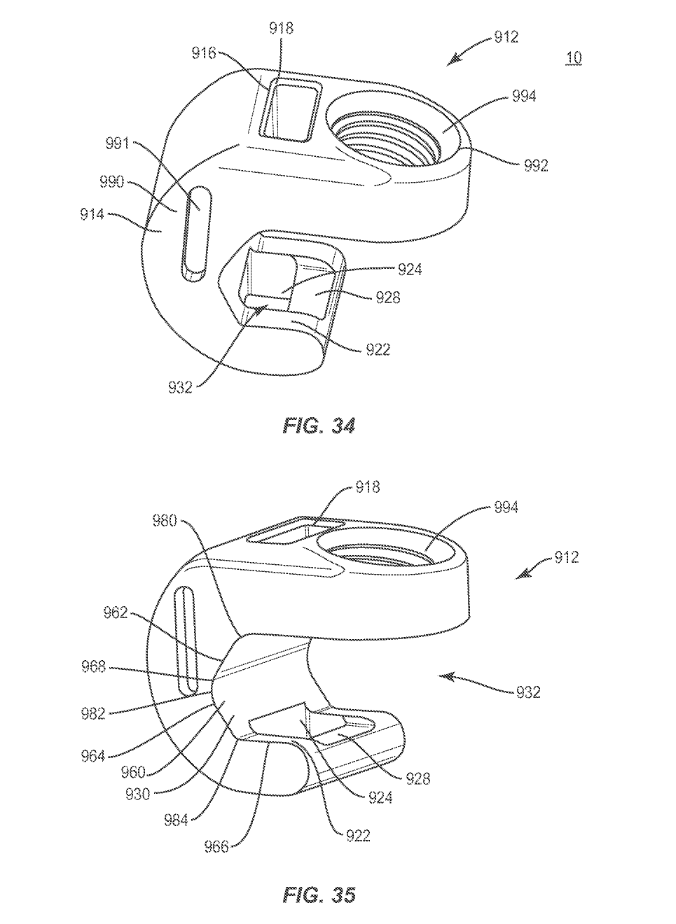

In one embodiment, as shown in FIGS. 34 and 35, spinal implant system 10, similar to the systems and methods described herein, comprises a connector 912, similar to connector 12 described herein. Connector 912 includes a body 914 having a surface 916 that defines an opening 918 configured for disposal of a member, such as, for example, a tether 20, as described herein.

Body 914 includes a surface 922 that defines an opening 924 configured for disposal of tether 20. A portion of surface 922 includes a groove, such as, for example, a cutout 928. Cutout 928 is configured to provide a flush engagement between tether 20 and surface 922 when tether 20 is disposed with opening 924.

Body 914 includes a surface 930 that defines a passageway 932. Passageway 932 has an oblong configuration and extends through body 914. Passageway 932 is configured for disposal of a longitudinal element, such as, for example, a spinal rod 54, such that connector 912 can be mounted with spinal rod 54, as described herein.

Surface 930 includes a wall 960 comprising a plurality of adjacent planar surfaces. Wall 960 includes planar surfaces, such as, for example, flats 962, 964, 966, similar to flats 62, 64, 66 described herein. Flats 962, 964, 966 define an engagement surface and/or a lock surface 968. In some embodiments, wall 960 defines a plurality of cavities between and adjacent flats 962, 964, 966 that prevent spinal rod 54 from undesirably engaging and/or pinching tether 20. Wall 60 includes a cavity, such as, for example, arcuate reliefs 980, 982, 984, similar to arcuate reliefs 80, 82, 84 as described herein.

Body 914 includes a mating surface 990 that defines cavities, such as, for example, mating capture elements 991 configured to mate with a surgical instrument to facilitate implant and manipulation of connector 912 and/or components of spinal implant system 10. Body 914 includes a surface 992 that defines an opening 994. Surface 992 is threaded and configured for disposal of a set screw 96, as described herein.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A spinal construct comprising:
a member extending between a first end and a second end defining an opening configured for movement of the first end therethrough and disposal of the longitudinal member about spinal tissue; and
a malleable element disposed with the first end.

2. A spinal construct as recited in claim 1, wherein the malleable element comprises a malleable lead connected with the first end and being movable through the opening.

3. A spinal construct as recited in claim 2, wherein the lead includes an outer surface comprising an average surface roughness in a range of approximately 4 to 16 micro inches.

4. A spinal construct as recited in claim 2, wherein the lead includes an inner surface defining an inner cavity configured for disposal of the first end.

5. A spinal construct as recited in claim 2, wherein the lead is fabricated from a material having a durometer in a range of approximately 30 A to 60 A under the ASTM D2240 type A scale.

6. A spinal construct as recited in claim 2, wherein the lead includes an arcuate configuration.

7. A spinal construct as recited in claim 2, wherein the lead includes a paddle configuration having a blunt tip.

8. A spinal construct as recited in claim 1, wherein the first end includes an inner surface that defines a cavity configured for disposal of the malleable element.

9. A spinal construct as recited in claim 1, further comprising a connector that defines a first opening and a second opening configured for disposal of the member, and including a wall that defines a passageway configured for disposal of a longitudinal element.

10. A spinal construct as recited in claim 9, wherein the connector further includes a pin disposed adjacent the second opening, the pin being configured for attachment with the member.

11. A spinal construct as recited in claim 9, further comprising a locking element engageable with the longitudinal element and the wall including at least one planar surface.

12. A spinal construct as recited in claim 11, wherein the at least one planar surface defines at least one lock surface for fixation of the member between the longitudinal element and the at least one planar surface.

13. A spinal construct as recited in claim 11, wherein the at least one planar surface includes a plurality of adjacent planar surfaces.

14. A spinal construct as recited in claim 11, wherein the at least one planar surface includes a first planar surface and a second planar surface disposed at an angular orientation relative to the first planar surface.

15. A spinal construct as recited in claim 11, wherein the wall includes at least one relief disposed adjacent the at least one planar surface.

16. A spinal construct as recited in claim 11, wherein the at least one planar surface includes a plurality of adjacent planar surfaces and further comprising a relief disposed between the adjacent planar surfaces.

17. A method for treating a spine, the method comprising the steps of:
delivering a spinal construct to a surgical site including vertebrae, the spinal construct including a connector that defines a passageway and a tether having a malleable element;
disposing a longitudinal dement with the passageway;
connecting the tether with the vertebrae; and
engaging a locking element with the longitudinal element to translate the longitudinal element for fixation of the tether between the longitudinal element and the connector.

18. A method for treating a spine as recited in claim 17, wherein the connector includes a pin configured for attachment with a loop of the tether.

19. A method for treating a spine as recited in claim 17, wherein the connector includes at least one planar surface that defines at least one lock surface for fixation of the tether between the longitudinal element and the at least one planar surface.

20. A spinal implant system comprising:
a connector defining a first opening and a second opening, the connector including a wall that defines a passageway and has at least one planar surface;
a tether having a malleable element and being disposable with the openings;
a spinal rod disposable with the passageway; and
a locking element engageable with the spinal rod.
